(19) United States
(12) Patent Application Publication
(10) Pub. No.: US 2014/0161846 A1
(43) Pub. Date: Jun. 12, 2014

(54) SUNSCREEN WITH COOLING AGENT

(71) Applicant: THE DIAL CORPORATION, Scottsdale, AZ (US)

(72) Inventors: Chris Luciow, Phoenix, AZ (US); Travis T. Yarlagadda, Phoenix, AZ (US)

(73) Assignee: The Dial Corporation, Scottsdale, AZ (US)

(21) Appl. No.: 13/710,990

(22) Filed: Dec. 11, 2012

(51) Int. Cl. A61K 8/11; A61Q 17/04

(52) U.S. Cl. CPC A61K 8/11; A61Q 17/04
USPC 424/401; 424/59

(57) ABSTRACT

A sunscreen formulation and methods for making the same include a sunscreen compound into which is added a cooling agent. The cooling agent will produce a cooling or cooling sensation when experienced by a user of the sunscreen. A controlled release mechanism is used to delay activation of the cooling agent.

### FIELD OF THE INVENTION

The present invention generally relates to sunscreen or sun-protection lotion, and more particularly relates to sun-protection lotion incorporating a cooling agent.

### BACKGROUND OF THE INVENTION

In all parts of the world, people enjoy outdoor activities. In such activities, any bare skin is exposed to radiation from the sun, particularly in the visible and ultraviolet (UV) wavelengths. The amount of radiation received on the skin will depend on factors such as the weather, the time of day and the length of exposure to the sun.

In numerous studies, it has been clearly demonstrated that exposure to the sun, particularly to the extent that a sunburn is received, causes various skin disorders, not the least of which is skin cancer. For this reason, it is widely advocated that people engaged in outdoor activities apply a protective lotion, also known as sunscreen or sunblock, on any exposed skin. Such lotions will absorb solar radiation, particularly in the ultra-violent wavelengths, which is primarily responsible for resultant sun burns. By absorbing the potentially dangerous radiation, the applied lotion protects the skin from harm or at least mitigates any damage done.

Different compositions of sunscreen lotion can absorb potentially harmful radiation to different degrees. Consequently, a scale has been developed in which the Sun Protection Factor (SPF) of a sunscreen lotion is numerically rated. The higher the number, the more that lotion will absorb potentially harmful solar radiation, particularly ultraviolet radiation, to protect the underlying skin.

However, even though a sunscreen may have a relative high SPF rating such that the skin is generally protected from the burning effects of solar radiation, thermal energy from the sun may still accumulate in an individual's exposed skin causing the skin to feel warm or even hot. Depending on the amount of sunlight received, this heat may become uncomfortable on the skin. This is particularly true if the person is involved in any physical activity or exercise in the sun, which will also generate body heat thereby increasing discomfort.

### BRIEF SUMMARY OF THE INVENTION

A sunscreen formulation comprises a sunscreen; a cooling agent mixed in the sunscreen compound; and a controlled release mechanism that delays contact between the cooling agent and a user's skin when the sunscreen formulation is applied to the user's skin.

A method of making a sunscreen formulation includes adding a cooling agent to said sunscreen compound; and providing a controlled release mechanism that delays activation of said cooling agent when said sunscreen formulation is applied by a user.

### BRIEF DESCRIPTION OF THE DRAWINGS

The accompanying drawings illustrate various examples of the principles described herein and are a part of the specification. The illustrated examples are merely illustrative and do not limit the scope of the claims.

FIG. 1 is an illustration of a sunscreen formulation comprises a controlled release mechanism governing a cooling agent according to one example of principles described herein.

FIG. 2 is an illustration of a sunscreen formulation comprises an encapsulant packaging a cooling agent according to one example of principles described herein.

FIG. 3 is a flowchart showing an illustrative formulation of a sunscreen according to one example of principles described herein.

FIG. 4 is a flowchart showing another illustrative formulation of a sunscreen according to one example of principles described herein.

FIG. 5 is an illustration of an encapsulant package of cooling agent for use in a sunscreen according to one example of principles described herein.

FIG. 6 is an illustration of an encapsulant package of cooling agent using an oleosome for use in a sunscreen according to one example of principles described herein.

Throughout the drawings, identical reference numbers designate similar, but not necessarily identical, elements.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

As noted above, even though a sunscreen may be used as protection from the burning effects of solar radiation, thermal energy from the sun may still accumulate in an individual's exposed skin causing the skin to feel uncomfortably warm or even hot. Thus the individual may be protected from the harmful UV rays when using sunscreens that are currently available, but can still be uncomfortable due to the heat of the sunlight.

To address this issue, a cooling agent can be added to the sunscreen that will cause the skin to feel cooler, despite the incident sunlight. However, such a sunscreen might have cooling properties that are uncontrolled. For example, the user may feel chilly when applying the sunscreen indoors or before they are outdoors in the sunlight. Consequently, the present specification discloses a sunscreen that protects from harmful UV rays and maintains the comfort of the individual when the individual is actually out in the sun.

To achieve this, a controlled-release mechanism can also be added to the sunscreen to delay the release of the cooling agent so that the cooling effect occurs over a prolonged period. For example, a mechanism that delays contact between a user's skin and the cooling agent will prolong the cooling effect or sensation achieved.

As used herein, the term “sunscreen” or “sunscreen formulation” will refer broadly to a composition that is effective to decrease the potentially harmful effects of solar radiation on a user's screen. This protective quality can be rated with a Sun Protection Factor (“SPF”). The term “sunscreen” will be used inclusive of all similar terms such as sunblock, suntan lotion, and the like. A sunscreen with an incorporated cooling agent may be formulated in, for example, lotions, creams, aerosols, and liquids delivered by pumps or sprays.

As used herein, the term “cooling agent” will refer broadly to an agent that can be incorporated into a sunscreen, where the cooling agent, when applied by a user, produces or facilitates either a cooling sensation or an actual reduction in thermal energy. A “cooling sensation agent” will refer more specifically to a cooling agent that produces a cooling sensation without necessarily have any actual effect on temperature.

As used herein, the term “controlled-release mechanism” will refer broadly to any mechanism, chemical or mechanical, that delays the effect of a cooling agent in an applied sunscreen. For example, the controlled-release mechanism may delay contact between the cooling agent and the skin of a user that has applied to a sunscreen incorporating the cooling agent. In this way, the activity of the cooling agent can be extended over a more prolonged period.

On example of a cooling agent for incorporation into a sunscreen is menthol. Menthol derivatives may also be used and include, but are not limited to, natural menthol, synthetic menthol, racemic menthol derivatives, menthol isomers including neomenthol, isomenthol, neoisomenthol, cornmint oil, racemic and isomers of synthetic menthol like cooling agents to include but not limited to monomenthol succinate (MMS), menthol ethylene glycol carbonate, menthol propylene glycol carbonate, menthone glycerol ketal, menthol lactate, 3-(1-menthoxy) propane 1-2 diol (MPD), Coolact agent 10, TK-10, 3-(1-menthoxy)-2-methylpropane-1-2-diol, 3-(1-menthoxy)ethanol (Coolact 5), 3-(1-menthoxy)propan-1-ol, 3-(1-menthoxy)-butan-1-ol, Isoupulegol (Coolact P), p-menthane-3,8 diols (Coolact 38D, PMD38), menthone glycerol ketal (Frescolat MGA), menthyl lactate (Frescolat ML), (2S)-3-(1-methoxy)-propane-1-2 diol (MPD, TK10), methyl-3-hydroxybuterate (MHB), menthyl pyrrolidin-2-one5-carboxylate (Questice), 6-Isopropyl-9-dimethyl-1-4-dioxaspiro [4,5]decan-2-one, Cubebol, DL-pyrrolidin-2-one carboxylic acid. Carboxamide cooling agents to include but not limited to alkyloxy amides of the p-menthane carboxamide series of coolant molecules, D-Ala-O-Me and D-Ala-O-Et, N-(R)-2-oxotetrahydrofuran-3-yl(1R,2S5R)-p-menthane-3carboxamide (D-HSL), N-ethyl-p-menthane-3-carboxamide, 2-isorpropyl-trimethylbutyarmide, N-([ethoxycarbonlyl)methyl]methyl)-p-methane-3-carboxamide, ethyl-(p-menthane-3-carboxamido)acetate, N,N-dimethyl menthyl succinamide, N-(2-ethoxyethyl)-2-isopropyl-2,3-dimethylbutanamide, and the like. Menthol and its derivatives are cooling sensation agents, as will be described in further detail below.

Additional examples of cooling sensation agents include, but are not limited to, vanillyl butyl ether, peppermint oil, methane carboxamide ethyl pyridine, menthoxypropanediol, menthanediol, cyanomethylphenyl menthane carboxamide, camphor, ethyl menthane carboxamide, menthyl diisopropyl propionamide, menthyl lactate, 4-(butoxymenthyl)-2-methoxy-phenol, 3-[[5-methyl-2-(1-methyl)cyclohexyl]oxy]-1,2-propanediol, isopulegol, or a mixture thereof. In one example, the cooling sensation agent is Winsense® Extra 500, which comprises a mixture of ethyl menthane carboxamide, menthyl diisopropyl propionamide and menthyl lactate. Winsense® Extra 500 is manufactured by LyondellBasell of Rotterdam, the Netherlands. Other suitable cooling sensation agents known to those skilled in the art may also be used.

As indicated, different cooling agents may employ different mechanisms to produce cooling or a cooling sensation on the skin. For example, cooling sensation agents, such as menthol and menthol derivatives will activate the protein TRPM8 or TRPA1 when coming into contact with the skin. This protein, the transient receptor potential cation channel subfamily M member 8 (TRPM8), also known as the cold and menthol receptor 1 (CMR1), when activated by the cooling agent, will produce a neuro-physiological signal to the central nervous system that is similar to the signal naturally produced by dermal nerve endings to indicate a cold temperature. Consequently, the user has the sensation of a cooling on the skin even though the skin temperature is not actually lowered.

In other possible examples, the cooling agent may promote the evaporation of perspiration from the skin. This will enhance the natural cooling effect of perspiration and actually lower the temperature of the skin. Any cooling agent that either produces a cooling sensation or actually reduced the temperature of the skin may be used in a sunscreen as disclosed herein.

Because outdoor activity is often prolonged, it is advantageous for the operation of the cooling agent to continue over an extended period of time. As indicated above, this may be accomplished by providing some controlled-release mechanism by which the cooling agent is released or made effective gradually over time.

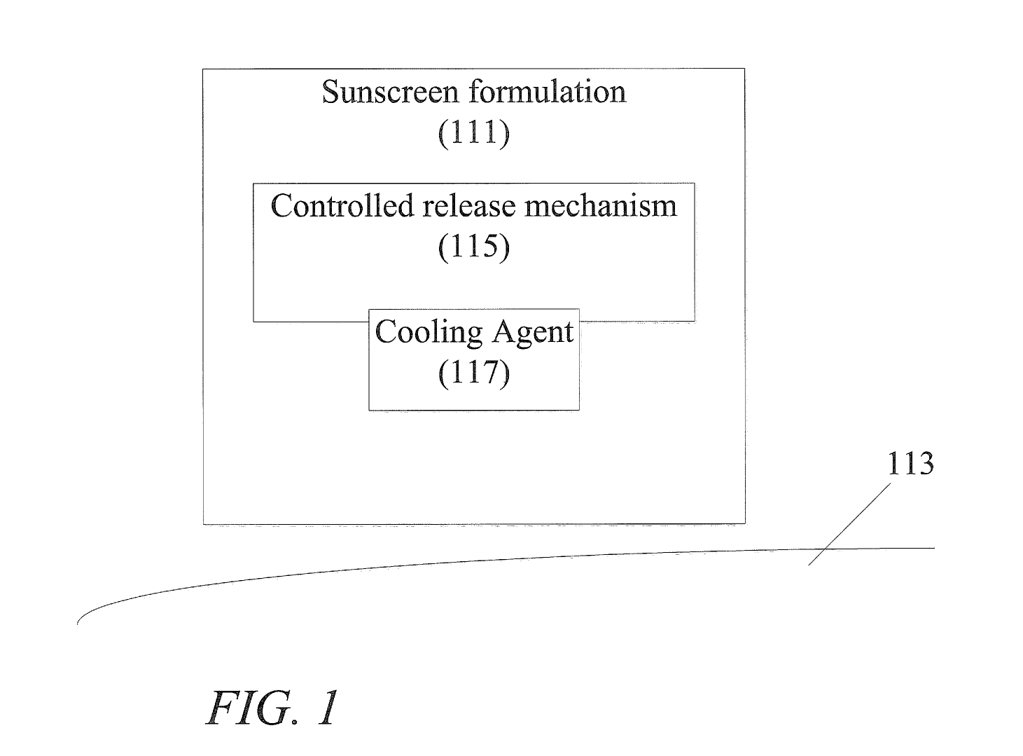

This concept is illustrated in FIG. 1. As shown in FIG. 1, a sunscreen formulation (111) incorporates a cooling agent (117). The cooling agent (117) is released or made effective subject to a controlled release mechanism (115). The cooling agent (117) produces a cooling effect, as described herein, for the user's skin (113) on which it is applied. Various examples of cooling agents and corresponding controlled release mechanisms will be described herein.

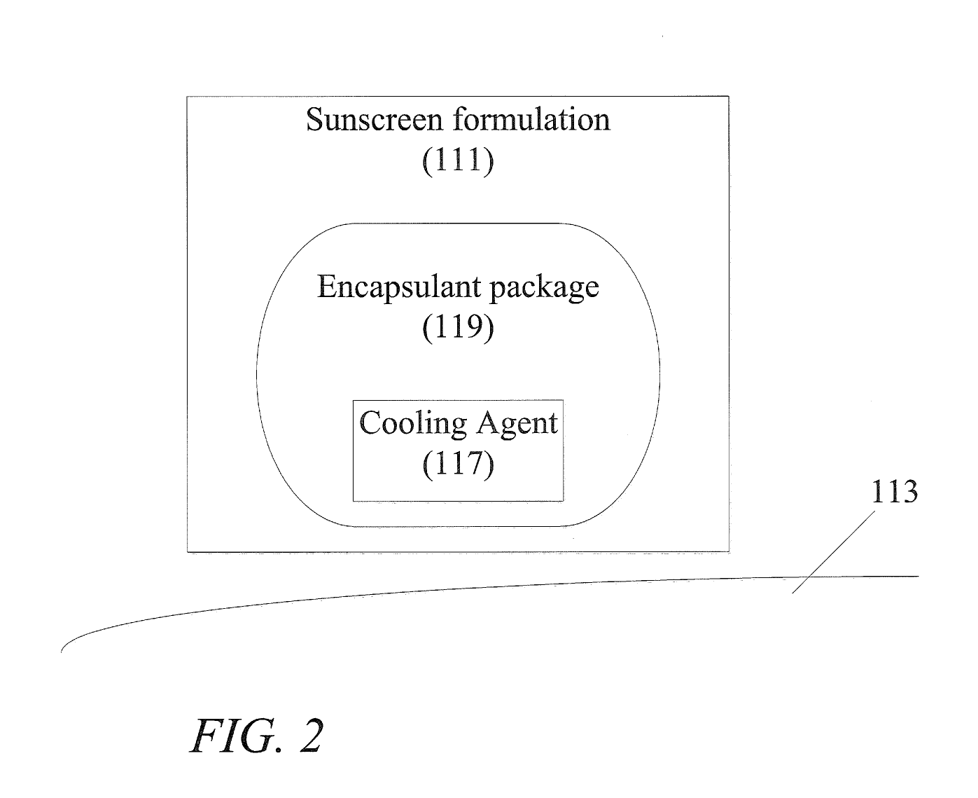

In FIG. 2, for example, the cooling agent (117) may be contained within an encapsulant package (119). As will be described below, this encapsulant package (119) may be, for example, a polymer shell, an oleosome, an absorbent silica particle, a photocleavable or photo-oxidizable encapsulant, or other encapsulant. The encapsulant package (119) may respond to, for example, moisture or radiation, so as to release the cooling agent (117). As the carrier or encapsulant gradually releasing quantities of the cooling agent to the user's skin (113) over an extended period, the cooling effect of the cooling agent is prolonged.

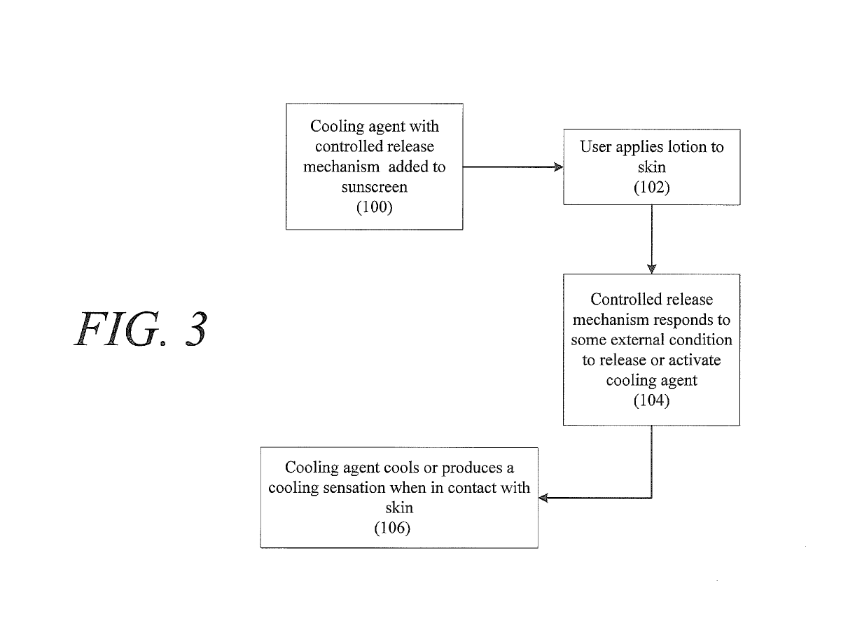

FIG. 3 is a flowchart showing an illustrative preparation in which a cooling agent is mixed into a sunscreen formulation with a controlled release mechanism that will allow quantities of the cooling agent to be held suspended in the sunscreen until the controlled release mechanism is subsequently triggered. The controlled release mechanism may also promote the shelf-life of the cooling agent in the sunscreen. When the controlled release mechanism is triggered, the cooling agent is activated or released and allowed to come into contact with the user's skin.

With the lotion so prepared, the user can apply the sunscreen to his or her skin for protection from harmful solar radiation (102). The SPF of the sunscreen may be independent of the presence of the cooling agent. Although, the presence of the cooling agent may have an impact on the effective SPF of the sunscreen. Thus, a sunscreen formulation with a cooling agent, as described herein, may be prepared with different levels of SPF to meet varying user demands.

Over the course of time, the trigger for the controlled release mechanism for the cooling agent will occur (104). Various examples are provided below of different controlled release mechanisms and the external condition or conditions which will trigger each to release′ or activate the cooling agent. In this way, the cooling agent may be released and become effective when it is most needed and over a prolonged period. When the cooling agent contacts the skin, it produces a cooling effect or sensation as described herein (106).

Specific examples of sunscreen with a cooling agent will now be described. Each of these examples is merely illustrative and the scope of the claims is not limited by or to any particular example.

### Example 1

In a first example, the cooling agent is incorporated in a carrier or encapsulated in an encapsulant that releases the cooling agent in the presence of moisture. Thus, perspiration, indicating that the user is feeling warm, or other moisture on the skin can release the cooling agent to produce the resulting cooling effect.

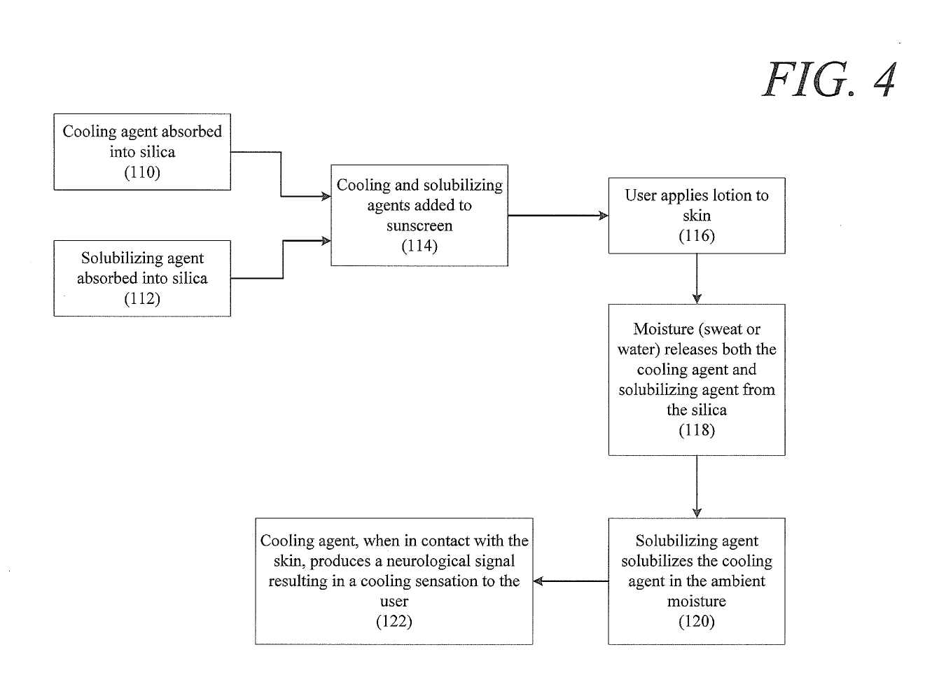

Within this first example, a specific formulation is shown in FIG. 4. As shown in FIG. 4, a cooling agent, for example, menthol or a menthol derivative, in liquid form, is absorbed into silica (110). The silica serves as a carrier and as a controlled release agent for the cooling agent over an extended time.

Additionally, a solubilizing agent, also in liquid form, is likewise absorbed into silica (112). Examples of solubilizing agents include, but are not limited to, polyglycol, polyethylene glycol, polypropylene glycol, or a mixture thereof. Other suitable solubilizers known to those skilled in the art may also be used. Again, the silica serves as a carrier and as a controlled release agent for the solubilizing agent over time.

The cooling agent, embedded in silica, and the solubilizing agent, embedded in silica, are both added to a sunscreen formulation (114). Subsequently, a user will apply this sunscreen to his or her skin for protection from solar radiation. When the applied sunscreen is later exposed to ambient moisture, presumably from the user's perspiration, that moisture will cause the release of the cooling and solubilizing agents from the silica carrier (118). If perspiration is the ambient moisture that releases the cooling and solubilizing agents from the silica carrier, the release of these agents will most likely coincide with the user feeling hot, as indicated by the presence of perspiration. However, exposure to water or other moisture may also release the cooling and solubilizing agents.

Specifically, the silica particles are porous and hydrophilic such that when the user perspires or other ambient moisture is introduced, the moisture is readily absorbed by the silica particles. This displaces or drives out the cooling agent and the solubilizer from the silica particles. The cooling agent and solubilizer are then able to interact. Once released, the solubilizing agent will solubilize the cooling agent in the ambient moisture that has caused the release of the two agents from the silica (120). This will allow the cooling agent to remain present in the moisture without being as readily washed away and to penetrate the moisture and other sunscreen constituents so as to contact the skin.

In this example, the cooling agent and the solubilizer are kept separately absorbed in different batches of silica particles. Consequently, the cooling agent and the solubilizer are combined only after being released from the silica by the introduction of ambient moisture. As a result, the sunscreen product has an enhanced shelf-life and provides the user with a cooling sensation effect over an extended period of time, likely when the user is perspiring and most desires a cooling sensation.

The solubilized cooling agent, when in contact with the skin, produces either a cooling sensation or an actual temperature reduction in the skin (122). In the present example, the cooling agent, being menthol or a menthol derivative, produces a neurological signal resulting in a cooling sensation to the user.

If the sunscreen in this example includes an oil/water (O/W) emulsion, the cooling and solubilizing agents will be incorporated in such a manner as to prevent early release from the water in the water phase. Both types of loaded hydrophilic grades of silica can be placed within the oil phase of the O/W emulsion. Another solution is to incorporate the cooling agents within a hydrophobic grade of silica within the water phase and the solubilizing agents incorporated within a hydrophilic grade of silica within the oil phase. Once sweat or water has reached the solubilizing agent silica, the solubilizing agents can slowly release the cooling agents from the hydrophobic grade of silica.

### Example 2

In a second example, the cooling agent is incorporated in a carrier or encapsulated in an encapsulant that is degraded by exposure to ultra-violet radiation, which is present in sunlight. For example, the cooling agent may be encapsulated in weakened polymer (hybrid) shells. Polymers shells comprised of low-density polyethylene or aromatic rings readily degrade upon prolonged exposure to ultra-violet radiation.

In this example, as the user's skin is exposed to sunlight, the ultra-violet component of the solar radiation will gradually degrade the carrier or encapsulant of the cooling agent. As this degradation progresses, the encapsulant will eventually release the cooling agent in a time-controlled manner.

In this example, the cooling agent may be stored in varying quantities of a carrier or encapsulant. Consequently, some of the cooling agent stored in a relatively lower amount of carrier or encapsulant is released first. An additional quantity of the cooling agent stored in a relative greater amount of carrier or encapsulant requires more ultra-violet radiation, and hence more time, before release. Any number of different levels of carrier or encapsulant may be used to store quantities of cooling agent within a sunscreen

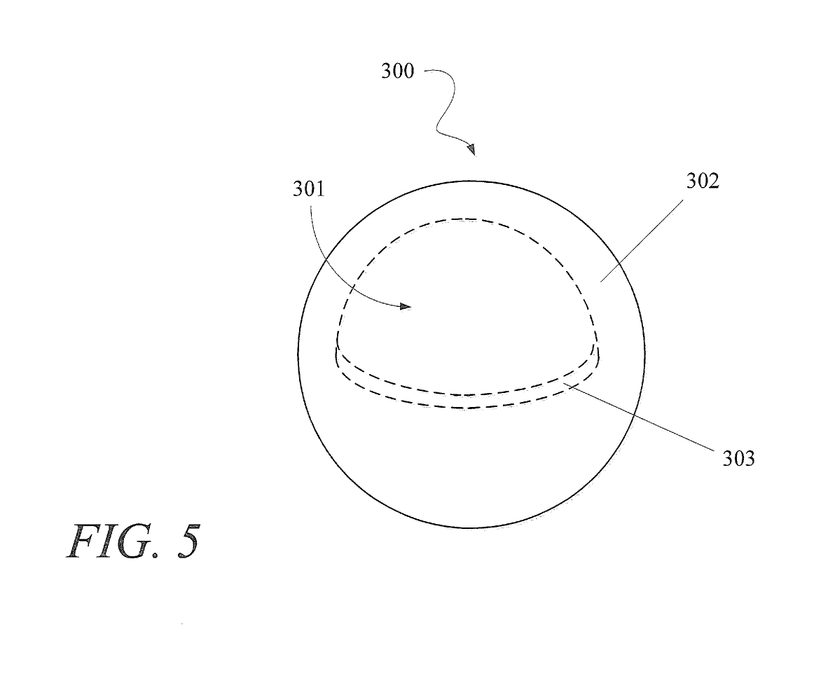

This is illustrated in FIG. 5. As shown in FIG. 5, a package (300) is provided in which a quantity of cooling agent (301) is encapsulated in an encapsulant shell (302). As indicated above, this encapsulant shell will degrade or disintegrate under exposure to ultraviolet light so as to release the cooling agent (301). The shell (302) has a thickness (303) that can vary from package to package (300). Because of this varying thickness, different shells (302) will naturally degrade and release their cooling agent payload at different rates. Specifically, it will take longer for a thicker shell (302) to degrade or disintegrate under the same amount of ultraviolet radiation as a thinner shell. Thus, again, because of this varying thickness, different shells (302) will degrade and release their cooling agent payload at different rates. Consequently, two or more separate batches of these packages (300) can be prepared with a different shell thickness. When packages (300) from these two or more batches are combined in a sunscreen formulation, the sunscreen formulation then has packages that release cooling agent at two or more different rates to further prolong the effect of the cooling agent. Any number of different shell thicknesses may be used in a sunscreen formulation.

A solubilizing agent may also be stored separately in a carrier or encapsulant. As indicated above, the solubilizing agent may stabilize the cooling agent in the presence of ambient moisture thereby promoting the efficacy of the cooling agent in contacting the skin as described above.

### Example 3

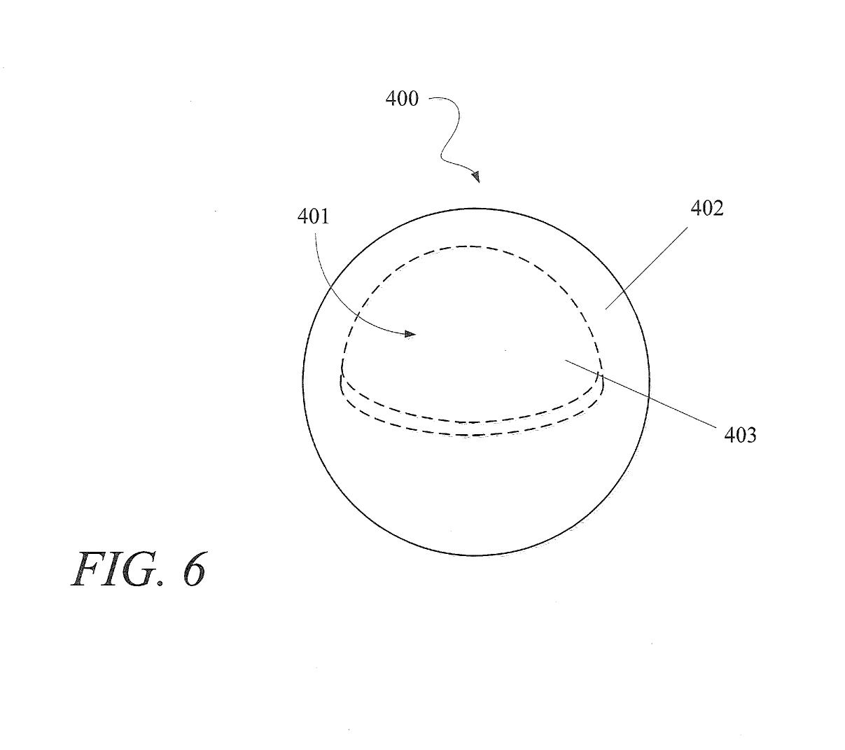

In another example, the cooling agent may be captured within oleosomes. This example will be illustrated with reference to FIG. 6.

Oleosomes are naturally-occurring microparticles (400) found within seeds, botanicals and nuts. These oleosomes consist of a core of vegetable oil and vitamin E (401) that is surrounded by a phospholipid membrane and protein coat (402). In contact with skin, the oleosome (400) breaks down and releases its contents. Where this content is the natural oil and vitamin E, the oleosome serves to deliver this as an emollient and emulsifier to the skin.

Additionally, oleosomes are capable of absorbing and delivering other substances, such as the cooling agent described herein. Simple mixing of oleosomes (400) with the desired cooling agent an result in the cooling agent (403) being loaded into and protected by the oleosome (400). The loaded olesomes (400) can then be added to a sunscreen.

When the oleosomes (400) come into contact with skin, they begin to dry out. Eventually, the outer membrane (402) disintegrates releasing the cooling agent (403) loaded within. This disintegration will naturally occur at variously different rates for different oleosomes depending on the oleosome composition and the ambient conditions immediately around the oleosome. Consequently, different oleosomes will disintegrate and deliver cooling agent at different times. This will further extend over time the cooling effect experienced by the user.

The following tables document two different illustrative sunscreen formulations in which oleosomes are used as the time-release delivery mechanism for a cooling agent.

| | Activity | Active | Blend Wt |
|---|---|---|---|
| Ingredient (INCI Name) | (%) | Wt (%) | (%) |
| Water (D.I) | 100.00 | Q.S. | 66.01 |
| Tetrasodium EDTA | 40.00 | 0.02 | 0.05 |
| Carbomer | 100.000 | 0.14 | 0.14 |
| Glycerin | 99.00 | 5.25 | 5.30 |
| Methylparaben | 100.00 | 0.25 | 0.25 |
| Cetyl Alcohol | 100.00 | 0.215 | 0.22 |
| Neopentyl Glycol Diheptanoate | 100.00 | 1.25 | 1.25 |
| Petrolatum | 100.00 | 1.00 | 1.00 |
| Glyceryl Stearate | 100.00 | 2.80 | 2.80 |
| Cyclopentasiloxane | 100.00 | 0.30 | 0.30 |
| Dimethicone | 100.00 | 0.50 | 0.50 |
| Propylparaben | 100.00 | 0.15 | 0.15 |
| Avobenzone | 100.00 | 3.00 | 3.00 |
| Homosolate | 100.00 | 7.50 | 7.50 |
| Octisalate | 100.00 | 5.00 | 5.00 |
| Octocrylene | 100.00 | 2.00 | 2.00 |
| Acrylates/C10-30 Alkyl Acrylate | 100.00 | 0.20 | 0.20 |
| Crosspolymer | | | |
| Aminomethyl Propanol | 100.00 | 0.182 | 0.18 |
| Phenoxyethanol | 100.00 | 0.55 | 0.55 |
| Tocpheryl Acetate | 100.00 | 0.10 | 0.10 |
| Menthol | 100.00 | 0.20 | 0.20 |
| Carthamus Tinctorius (Safflower) | 100.00 | 3.00 | 3.00 |
| Oleosomes | | | |
| Fragrance | 100.00 | 0.30 | 0.30 |
| | Totals = | 33.9 | 100.00 |

Bulk Release Limits

pH = 5.60-6.75
Viscosity (cps) = 120,000-280,000
SG = 1.005
Viscosity Method: Brookfield, T-F, 3 RPM

| % Actives | Bulk Release Limits | | |
|---|---|---|---|
| INN Name | Min | Target | Max |
| Avobenzone = | 2.75 | 3.00 | 3.25 |
| Homosolate = | 7.01 | 7.50 | 7.99 |
| Octisalate = | 4.60 | 5.00 | 5.40 |
| Octocrylene = | 1.84 | 2.00 | 2.16 |
| pH = | 5.60 | 6.12 | 6.75 |
| Microquality = | Same release limits as other | | |
| | liquid PC products | | |
| Preservative | Same release limits as other | | |
| Effectiveness = | liquid PC products | | |
| % Actives | Shelf Life Release Limits | | |
| INN Name | Min | Target | Max |
| Avobenzone = | 2.70 | 3.00 | 3.50 |
| Homosolate = | 6.75 | 7.50 | 8.70 |
| Octisalate = | 4.50 | 5.00 | 5.80 |
| Octocrylene = | 1.80 | 2.00 | 2.35 |
| pH = | 5.50 | 6.12 | 6.85 |

This example may be prepared by warming the oleosomes to 110° F. and mixing in the menthol. The menthol is then absorbed into the oleosomes as described above. The loaded oleosomes are then added the sunscreen formulation. The formulation may also optionally include a fragrance as indicated.

TABLE II

| | | Active | Blending |
|---|---|---|---|
| Ingredient (INCI) | Activity (%) | Wt (%) | Weight (%) |
| Water (D.I) | 100.00 | Q.S. | 80.89 |
| Glycerin | 99.00 | 5.35 | 5.40 |
| Tetrasodium EDTA | 40.00 | 0.02 | 0.05 |
| Methylparaben | 100.00 | 0.20 | 0.20 |
| Stearic Acid | 99.00 | 2.00 | 2.02 |
| Cetyl Alcohol | 100.00 | 0.75 | 0.75 |
| Ethylhexyl Palmitate | 100.00 | 1.75 | 1.75 |
| Propylene Glycol | 100.00 | 0.30 | 0.30 |
| Dicaprylate/Dicaprate | | | |
| Petrolatum | 100.00 | 1.20 | 1.20 |
| Glyceryl Stearate | 100.00 | 1.20 | 1.20 |
| Glycol Stearate | 100.00 | 1.20 | 1.20 |
| Dimethicone | 100.00 | 0.50 | 0.50 |
| Propylparaben | 100.00 | 0.10 | 0.10 |
| Carbomer | 100.00 | 0.14 | 0.14 |
| Cyclopentasiloxane | 100.00 | 0.30 | 0.30 |
| Sodium Hydroxide | 50.00 | 0.098 | 0.196 |
| Diazolidinyl Urea | 100.00 | 0.30 | 0.30 |
| Menthol | 100.00 | 0.20 | 0.20 |
| Carthamus Tinctorius (Safflower) | 100.00 | 3.00 | 3.00 |
| Oleosomes | | | |
| Fragrance | 100.00 | 0.30 | 0.30 |

This example may also be prepared by warming the oleosomes to 110° F. and mixing in the menthol. The menthol is then absorbed into the oleosomes as described above. The loaded oleosomes are then added the sunscreen formulation. The formulation may also optionally include a fragrance as indicated.

### Example 4

In still another example, the cooling agent could be encapsulated in aromatic aldehydes. This encapsulant that will be photocleavable or experience photooxidation with Norrish (Type II) reactions.

In organic chemistry, the Norrish reaction describes a photochemical reaction that take place with ketones and aldehydes. A Norrish type II reaction is the photochemical intramolecular abstraction of a γ-hydrogen, i.e., a hydrogen atom three carbon positions removed from the carbonyl group, by an excited carbonyl compound to produce a 1,4-biradical as a photoproduct.

Consequently, when exposed to sunlight, this encapsulant will also disintegrate. As with other examples herein, this will release the encapsulated cooling agent. This will allow the cooling agent to be delivered when it is most needed, i.e., upon exposure to sunlight.

### Example 5

An another example, the cooling agent may be solubilized in the oil phase of an oil/water emulsion within the sunscreen lotion. In this example, when the lotion is applied and the user subsequently perspires or encounters moisture, the emulsion breaks as a result of the added moisture. Consequently, the cooling agent, freed from emulsion, is able to reach the skin and produce an actual cooling or cooling sensation as described above.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

In another example, the cooling agent may be provided in the sunscreen in a compressed or pellet form. In the presence of moisture, such as perspiration, the cooling agent structure may begin to disintegrate thereby releasing amounts of cooling agent that are then effect to produce the desired cooling sensation.

The following table provides an illustrative formulation for according to this example. In this formulation, the cooling agent is menthol in pellet form.

TABLE III

| | Activity | Active | Blend |
|---|---|---|---|
| Ingredient (INCI Name) | (%) | Wt (%) | Wt (%) |
| Water (D.I) | 100.00 | Q.S. | 69.01 |
| Tetrasodium EDTA | 40.00 | 0.02 | 0.05 |
| Carbomer | 100.000 | 0.14 | 0.14 |
| Glycerin | 99.00 | 5.25 | 5.30 |
| Methylparaben | 100.00 | 0.25 | 0.25 |
| Cetyl Alcohol | 100.00 | 0.215 | 0.22 |
| Neopentyl Glycol | 100.00 | 1.25 | 1.25 |
| Diheptanoate | | | |
| Petrolatum | 100.00 | 1.00 | 1.00 |
| Glyceryl Stearate | 100.00 | 2.80 | 2.80 |
| Cyclopentasiloxane | 100.00 | 0.30 | 0.30 |
| Dimethicone | 100.00 | 0.50 | 0.50 |
| Propylparaben | 100.00 | 0.15 | 0.15 |
| Avobenzone | 100.00 | 3.00 | 3.00 |
| Homosolate | 100.00 | 7.50 | 7.50 |
| Octisalate | 100.00 | 5.00 | 5.00 |
| Octocrylene | 100.00 | 2.00 | 2.00 |
| Acrylates/C10-30 Alkyl | 100.00 | 0.20 | 0.20 |
| Acrylate Crosspolymer | | | |
| Aminomethyl Propanol | 100.00 | 0.182 | 0.18 |
| Phenoxyethanol | 100.00 | 0.55 | 0.55 |
| Tocpheryl Acetate | 100.00 | 0.10 | 0.10 |
| Menthol | 100.00 | 0.20 | 0.20 |
| Fragrance | 100.00 | 0.30 | 0.30 |
| | Totals = | 30.9 | 100.00 |

This example also includes the option of adding a fragrance to the sunscreen. The illustrated sunscreen may have an SPF of 15, but may also be applied to formulations with a different SPF.

The preceding description has been presented only to illustrate and describe embodiments and examples of the principles described. This description is not intended to be exhaustive or to limit these principles to any precise form disclosed. Many modifications and variations are possible in light of the above teaching.

1. A sunscreen formulation comprising:
a sunscreen compound;
a cooling agent mixed with said sunscreen compound;
a solubilizing agent that solubilizes the cooling agent in water; and
a controlled release mechanism that delays contact between said cooling agent and a user's skin, and also delays contact between the solubilizing agent and the user's skin, when said sunscreen formulation is applied to said user's skin, wherein the cooling agent is released subject to the controlled release mechanism, and the solubilizing anent is also released subject to the controlled release mechanism.

2. The sunscreen formulation of claim 1, wherein said cooling agent comprises menthol or a menthol derivative.

3. (canceled)

4. The sunscreen formulation of claim 1, wherein said controlled release mechanism comprises a carrier that absorbs said cooling agent and releases said cooling agent in response to the presence of moisture.

5. The sunscreen formulation of claim 4, wherein said carrier comprises silica.

6. The sunscreen formulation of claim 1, wherein said controlled release mechanism comprises an encapsulant that is degraded by exposure to ultra-violet radiation to release said cooling agent.

7. The sunscreen formulation of claim 6, wherein said encapsulant comprises a polymer shell.

8. The sunscreen formulation of claim 6, wherein quantities of said cooling agent are encapsulated in encapsulant shells of varying thickness such that said shells degrade and release said cooling agent at different rates due to said variation in shell thickness.

9. The sunscreen formulation of claim 1, wherein said controlled release mechanism comprises oleosomes into which said cooling agent is absorbed.

10. The sunscreen formulation of claim 1, wherein said controlled release mechanism comprises a photocleavable or photo-oxidizable encapsulant that encapsulates quantities of said cooling agent.

11. The sunscreen formulation of claim 10, wherein said encapsulant comprises aromatic aldehydes.

12. The sunscreen formulation of claim 1, wherein said controlled-release mechanism comprises solubilizing the cooling agent in an oil phase of an oil/water emulsion within a sunscreen such that said emulsion breaks in response to added moisture.

13. A method of making a sunscreen formulation comprising:
adding a cooling agent to said sunscreen compound; and
providing a controlled release mechanism that delays activation of said cooling agent when said sunscreen formulation is applied by a user.

14. The method of claim 13, wherein said cooling agent comprises menthol or a menthol derivative.

15. The method of claim 13, further comprising adding a solubilizing agent, wherein said solubilizing agent is also released subject to said controlled release mechanism.

16. The method of claim 13, wherein said controlled release mechanism comprises a carrier that absorbs said cooling agent and releases said cooling agent in response to the presence of moisture.

17. The method of claim 13, wherein said controlled release mechanism comprises encapsulating discrete quantities of said cooling agent in an encapsulant that is degraded by exposure to ultra-violet radiation to release said cooling agent.

18. The method of claim 17, wherein quantities of said cooling agent are encapsulated in encapsulant shells of varying thickness such that said shells degrade and release said cooling agent at different rates due to said variation in shell thickness.

19. The method of claim 13, wherein said controlled release mechanism comprises oleosomes into which said cooling agent is absorbed.

20. The method of claim 13, wherein said controlled release mechanism comprises a photocleavable or photo-oxidizable encapsulant that encapsulates quantities of said cooling agent.

21. The method of claim 13, wherein said controlled-release mechanism comprises solubilizing the cooling agent in an oil phase of an oil/water emulsion within a sunscreen such that said emulsion breaks in response to added moisture.
